(19) United States
(12) Patent Application Publication
(10) Pub. No.: US 2021/0330790 A1
(43) Pub. Date: Oct. 28, 2021

(54) Method for Treating Vitiligo Based on Enhancing the Alternative Cellular Energy Pathway Using KELEA Activated Water

(71) Applicants: William John Martin, South Pasadena, CA (US); Shera Sandwall, San Marcos, CA (US)

(72) Inventors: William John Martin, South Pasadena, CA (US); Shera Sandwall, San Marcos, CA (US)

(73) Assignee: MI Hope Inc, South Pasadena, CA (US)

(21) Appl. No.: 16/858,607

(22) Filed: Apr. 25, 2020

(51) Int. Cl. A61K 41/00; A61P 17/00

(52) U.S. Cl. CPC A61K 41/00; A61P 17/00

(57) ABSTRACT

Water can either naturally contain or can be made to contain an added level of an environmental force called KELEA, an abbreviation for Kinetic Energy Limiting Electrostatic Attraction. Placing KELEA activated water directly onto the area of skin with vitiligo leads to re-pigmentation of the affected area of skin. The therapy is attributed to enhancing the body's alternative cellular energy (ACE) pathway. This pathway has an anti-inflammatory effect and also a regenerative effect referred to as REACH, an abbreviation for Regenerative Energy Assisted Clinical Healing. The restoring of melanocyte in patients with vitiligo will likely apply to other illnesses in which there is melanocyte degeneration, including Parkinson's disease. Enhancing the ACE pathway using KELEA-activated water is also applicable to other illnesses classified as being autoimmune. The KELEA activated water can also be used in wearable waterceutical pouches.

### RELATED PUBLICATION BY THE APPLICANT

Martin W J (2020) Enhancing the Alternative Cellular Energy (ACE) Pathway with KELEA Activated Water as Therapy for Infectious Diseases. Infectious Disorders—Drug
Targets. February 10. doi: 10.2174/1871526520666200211115111.

### PENDING PATENT APPLICATIONS

Method of Enhancing the Alternative Cellular Energy Pathway in Humans and Animals Using Wearable Items That Contain KELEA Activated Water. Submitted Feb. 18, 2019, application Ser. No. 16/278,712
Method of Detection and Measurement of a Life Force Energy, Also Known as KELEA in Liquids & Other Materials. Submitted May 23, 2019, application Ser. No. 16/421,344 Using paper with heightened KELEA to Enhance the alternative cellular energy pathway. Submitted Jul. 10, 2019, application Ser. No. 16/508,255

### STATEMENT REGARDING FEDERALLY SPONSORED RESEARCH OR DEVELOPMENT

Not applicable: No Federal funding was received in support of this patent application.

### FIELD OF THE INVENTION

The invention is within the field of cellular energy-based therapies for inflammatory disorders and more specifically for use in the cure of vitiligo.

### BACKGROUND

Vitiligo is a disease characterized by the loss of melanin pigment in one or more circumscribed areas of the skin. The term melanin is derived from the Greek word melanos, meaning dark. The basic light-absorbing compounds comprising melanins are polymers of oxidized phenols, including quinones, with mineral binding capacities. The phenol is generally derived from the amino acid tyrosine. The extent of polymerization and other structural changes results in a diverse array of compounds included within the term melanins. Different types of melanins are distributed throughout evolution, including being present in some unicellular prokaryotic and eukaryotic organisms, and in all fungi, plants and animals. In humans, melanin is responsible for skin and hair pigmentation, with marked differences in its levels in different racial groups. The skin melanin is synthesized by neural-derived cells called melanocytes. The produced melanin is also distributed to neighboring keratinocytes and to hair follicles. Melanin is also present in other body sites, including the eye, inner ear, meninges, and the substantia nigra in the brain.

The localized absence of skin melanin in one or more areas of the skin is an acquired illness referred to as vitiligo, from the Latin word “vitellus” meaning veal, and referring to the paleness of the skin. Vitiligo is distinguished from the overall lack of skin pigmentation leading to albinism. Vitiligo affects approximately 0.5 to 1% of the human population. It can be localized to only a single small patch of skin or be more widely distributed as multiple non-pigmented patches on the body. Vitiligo is particularly noticeable in those of African heritage and especially when it involves the face.

The primary cause of vitiligo is attributed to the loss of melanocytes in the affected skin areas, rather than the failure of existing melanocytes to produce melanin. The loss of melanocytes is variously attributed to a primary immunological or auto-immune disease or, alternatively, to some other melanocyte destroying process, with a secondary immune (inflammatory) component. The patchy nature of the illness is difficult to explain by a systemic immune disorder, suggesting that there must be localized factors leading to the pimary degeneration of melanocytes. Still, certain pharmaceutical drugs can also induce localized vitiligo in susceptible individuals. Melanin can both protect cells from reactive oxygen species and can also be a source of reactive oxygen species, which if excessive can kill the cell. Virus infections are also occasionally considered as a possible contributory factor to vitiligo with emphasis given to human herpesviruses. There is an analogy with herpes zoster virus induced shingles, especially when the vitiligo is segmented and only affects a unilateral area of the skin corresponding to a particular nerve supply.

Vitiligo can be accompanied by major psychiatric symptoms, including depression, anxiety and low self-esteem. These are generally seen as secondary consequences of the stigma and unsightliness of the illness. It is likely, however, that emotional stress plays a major contributory role in the illness. Genetic factors have also be implicated in the disease.

Up to now, there has been no consistently successful method for treating vitiligo. Ultraviolet radiation is commonly used to induce melanin production with the optional addition of various photosensitizers, such as psoralens. Several natural products can lead to the overproduction of tyrosinase-related proteins through the Janus Kinase (JAK) pathway. These and more empirically-based natural compounds are being used clinically. The grafting of normally pigmented skin into the affected areas is also been tried. The range of current therapies underscores their overall ineffectiveness.

The inventors have achieved success in the treatment of vitiligo based on the underlying premise that it is likely caused by infection of the melanocytes with stealth adapted viruses and is recoverable by enhancing the alternative cellular energy (ACE) pathway. As described in the referenced article, (included herein by reference), the ACE pathway is expressed as an added dynamic or kinetic property of the body's fluids. It is maintained by the continuing absorption into the body of an environmental force called KELEA, an abbreviation for Kinetic Energy Limiting Electrostatic Attraction. This energy can also be absorbed into water, which may then serve as an additional source of KELEA to enhance the body's ACE pathway. These topics are also discussed in the above listed pending patent applications, which are included herein by reference.

### SUMMARY OF THE INVENTION

Recovery from vitiligo is achievable by supplying additional cellular energy to the affected areas of skin by soaking the affected areas in KELEA activated water. This was shown using a natural source of KELEA activated water available in a therapeutic soaking pond that is also rich in sediment from dissolved petrified ancient trees and has lush growing algae. The pond therapy also leads to remarkable reduction in stress and improved interpersonal interactions.

### DETAILS OF THE INVENTION

Without being a requirement for this invention, many chronic illnesses are attributed to infections with stealth adapted viruses. These viruses differ from the viruses from which they are derived in not producing inflammation. This is due to the deletion or mutation of the genes coding for the relatively few virus components that are normally targeted by the cellular immune system. Although not formally presented, one of the inventors has obtained positive stealth adapted virus cultures from several patients with vitiligo. The patients also had a range of neurological and psychiatric symptoms consistent with a stealth adapted virus encephalopathy. Even though the cellular immune system generally fails to engage stealth adapted viruses, there can still be cellular recovery from infection. This can be observed in patients and in virus inoculated animals. It can also be seen in the virus cultures. A striking feature of the virus infected cells is the marked disruption of the cells' mitochondria, the major source of cellular energy from the metabolism of food. Rather the cells produce pigmented, metal-binding, self-aggregating materials as an added source of cellular energy. They are referred to as ACE pigments. ACE pigments, as well as a wide range of dipolar, self-aggregating materials, can attract KELEA from the environment and transfer it to nearby water. This leads to a loosening of the hydrogen bonding between water molecules with each other and with dissolved electrolytes, including chloride ions. KELEA energy in water can be converted to chemical energy. Moreover, if water is sufficiently activated, it can transfer KELEA to nearby water, probably in an oscillatory manner. It can also transfer KELEA to human body fluids. This transfer can seemingly directly improve the brain's KELEA attracting ability leading to a sustainable increase in the body's ACE pathway.

KELEA activated water has increased volatility and lower surface tension due to the lowering of the intermolecular hydrogen bonding. It also displays far greater internal movements of added materials sprinkled onto the water. KELEA activated water can also be measured by increasing the measured weight of cellulose containing materials, including paper.

A natural source of KELEA activated water was identified as coming from a deep (>800 feet) well in a ranch in San Marcos, Calif. Some of the water was repeatedly applied to a somewhat barren, stone-laden hilltop within the ranch. The bucket on a frontend bulldozer broke while trying to move some of the embedded rock material, later identified as petrified ancient oak trees. This area was used to create a pond. Over the ensuring 12 years, the underlying solid material has been broken down into a granular, mud-like material. A lush growth of algae is on the surface of the pond. Fresh well water is periodically added to the pond. Rescued and abandoned horses brought onto the ranch were noted to commonly drink from the pond with obvious benefits to their health. This use was extended to the daily use by a very sick human, with major improvements in her health. There are now many additional examples of individuals recovering from chronic illnesses by either soaking in the pond or directly bathing in the well water. These will be described in further patent applications. This particular application is intended to indicate the remarkable benefits of soaking in the pond in the rapid disappearance of vitiligo.

A middle-age African American woman was disfigured by vitiligo, which extensively affected both cheeks on her face. It had been present for several years. She was coerced to spend approximately three hours a day for three days with her legs in the water of the pond. During these periods, she was instructed to repeatedly apply the water and granular mud-like material to her cheeks. Even at the end of the first day, an increase in coloration in her cheeks was noted. It became more obvious after the second day. By the end of the third day, the normal level of facial pigmentation was restored. The personality of the woman also markedly improved from being irritable and somewhat antagonistic to the ranch owner to being joyful and clearly more sociable.

In a second example, a Hispanic woman was disfigured with vitiligo affecting the lower half of her lower lip. She also showed extensive areas of depigmentation on the back of her left hand. She was invited to spend time in the pond. It also only required spending a few hours each day for 3 days for both her lower lip and her hand to appear normal. Restoration of the color of her lower lip along with an overall improvement in her facial complexion resulted in her appearing to be far younger than it was at first believed. Indeed, the gentleman who had accompanied the woman to the ranch was initially believed to be her son. With the regained normal facial appearance, it was apparent that he was her husband. Both of the treated women were hugely impressed and joyful with the results. They were each invited to have repeat treatments should the vitiligo ever return. Neither of the women ever did!

The recovery process from vitiligo is attributed to a combination of three factors. First, suppression of stealth virus infection if indeed this is the primary cause of the vitiligo. Second, an anti-inflammatory effect presumably evoked by the destruction and degeneration of the melanocytes. Third, the regeneration of new melanin-producing melanocytes. The anti-inflammatory effect of KELEA activated water is explained by added resilience and increased threshold needed to trigger the release and to response to inflammatory cytokines. The regenerative effect of KELEA activated water are embodied in the term REACH, an abbreviation for Regenerative Energy Assisted Clinical Recovery.

Although specifically describing the recovery from vitiligo, the thesis of this patent application has many broad applications. Melanin deficiency can affect many other processes in the body. Parkinson's disease is one example in which there is degeneration of melanin containing cells in the substantia nigra. Individuals with Parkinsonism have benefited from spending time in the pond. Melanin can also contribute to the inner ear and to the eyes. In a broader sense, vitiligo is generally grouped along with a wide range of autoimmune diseases. Combining anti-inflammatory and tissue regenerative effects can potentially lead to the recovery from many types of chronic illnesses.

The pond is specifically referred to in this application as a preferred environment to experience the benefits of KELEA activated water. It has the advantage of prolonged localized exposure over several hours, which is difficult to achieve using a skin spray or merely drinking the water. The presence and use of degraded petrified, mud-like material adds to the ambiance of the experience. There is no reason, however, not to believe that the water itself can significantly enhance the body's ACE pathway. Evidence accumulating over the last year is pointing to many benefits achievable by using KELEA activated water in wearable waterceutical Pouches™ that can be directly applied to the skin. These benefits are now also routinely being seen in horses to which the pouches are being applied after exercise.

It is understood, therefore, that the described therapy for vitiligo is applicable to the broader treatment of illnesses in which there can be an insufficiency of cellular energy. The Application can apply not only to the naturally available KELEA activated water, but to water in which an elevated level of KELEA can be achieved using various methods. These methods include the addition of dipolar, mineral-containing materials, such as humic and fulvic acids, zeolites, finely grounded and heated volcanic rock, mica, oxides of magnesium, germanium and other minerals, herbal tinctures, certain pharmaceuticals, such as vitamin C, Dilantin, procaine and lidocaine, gases including hydrogen, ozone, and chlorine dioxide, and other natural substances KELEA can also be added to water using electrolysis and various oscillating and fluctuating electrical devices. A major advantage of the naturally available KELEA activated water is that both the water from the well and the water in the bond have retained their activity from their first recognition 12 years ago of their having both healing powers and increased kinetic activity.

1. A method for treating vitiligo, comprising the repeated and prolonged application of water, which has absorbed an added level of the environmental force called KELEA, being an abbreviation for Kinetic Energy Limiting Electrostatic Attraction, to the area of skin affected with vitiligo in a manner that will lead to the re-pigmentation of the affected area of skin.

2. The method of claim 1 in which the KELEA activated water is used in the setting of a natural earth pond containing the KELEA activated water and in which the individual can repeatedly soak the vitiligo affected area of skin with the water from the pond.

3. A method for treating the psychiatric symptoms, which are commonly present in patients with vitiligo, comprising the spending of several hours soaking parts of the body in water, which has absorbed an added level of an environmental force called KELEA, being an abbreviation for Kinetic Energy Limiting Electrostatic Attraction, in a manner that will lead to a reduction of the psychiatric symptoms.

4. The method of claim 1 in which the KELEA activated water is contained in a wearable pouch, which can be placed onto the area of skin affected with vitiligo, for a period of time that is sufficient to lead to the re-pigmentation of the affected area of skin.

5. A method for treating diseases classified as being caused by an autoimmune response, comprising the use of water, which has absorbed an added level of the environmental force called KELEA, being an abbreviation for Kinetic Energy Limiting Electrostatic Attraction, in which the water is contained in a wearable pouch, which can be placed onto the area of skin of the individual affected with the autoimmune disease, for a period of time that is sufficient for the regression in the severity of the autoimmune disease.

6. The method of claim 1 in which the water being used has a naturally high level of KELEA.

7. The method of claim 1 in which the level of KELEA in the water being used has been increased using one or more dipolar compounds.

8. The method of claim 1 in which the level of KELEA in the water being used has been increased using one or more oscillating or fluctuating electrical devices

9. The method of claim 5 in which the water being used has a naturally high level of KELEA.

10. The method of claim 5 in which the level of KELEA in the water being used has been increased using one or more dipolar compounds.

11. The method of claim 5 in which the level of KELEA in the water being used has been increased using one or more oscillating or fluctuating electrical devices.
